(19) United States
(12) Patent Application Publication
(10) Pub. No.: US 2008/0200808 A1
(43) Pub. Date: Aug. 21, 2008

(54) DISPLAYING ANATOMICAL PATIENT STRUCTURES IN A REGION OF INTEREST OF AN IMAGE DETECTION APPARATUS

(76) Inventors: Martin Leidel, Miesbach (DE); Fritz Vollmer, Munich (DE); Ingmar Theimann, Munich (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/031,470

(22) Filed: Feb. 14, 2008

Related U.S. Application Data
(60) Provisional application No. 60/891,787, filed on Feb. 27, 2007.

(30) Foreign Application Priority Data
Feb. 15, 2007 (EP) 07003209

(51) Int. Cl. A61B 8/00

(52) U.S. Cl. 600/443; 600/454

(57) ABSTRACT

A method and device for detecting and displaying an anatomical patient structure in a region of interest of a movable image detection apparatus such as an ultrasound probe. After the region of interest is defined in a patient coordinate system, movement of the image detection apparatus can be tracked, and the position of the region of interest can be changed or shifted to compensate for the movement of the image detection apparatus.

### RELATED APPLICATION DATA

This application claims priority of U.S. Provisional Application No. 60/891,787 filed on Feb. 27, 2007, and EP 07003209 filed on Feb. 15, 2007, which are incorporated herein by reference in their entirety.

### FIELD OF THE INVENTION

The present invention relates to a method and system for displaying an anatomical patient structure in a region of interest of a movable image detection apparatus.

### BACKGROUND OF THE INVENTION

Ultrasound Doppler flow images are normally shown in a rather small part of a standard B-mode ultrasound image. The user can define a region of interest (ROI) relative to the image coordinates, i.e., relative to a coordinate system of an ultrasound head, wherein flow information is displayed, color-coded, in this region. The region of interest is a region in which an image detection device can detect particular properties of the patient structure.

When the ultrasound probe (head) is moved, the region of interest maintains its position within the image, since the region of interest is defined in relation to the coordinate system of the probe. Due to the movement of the probe, however, the anatomical structure to be displayed (for example, a blood vessel) can move out of the region of interest, and the user has to manually redefine a new region of interest. The user can manually redefine the region of interest by using an input on the hardware or software of the ultrasound apparatus.

This situation can be illustrated by way of FIGS. 1a and 1b. FIG. 1a shows a first ultrasound recording. Three vessels 11, 12 and 13, shown here by way of example, lie in an ultrasound detection plane 14, wherein the vessel 12 is shown along its progression and the vessels 11 and 13 are shown in cross-section. The user may manually define a region of interest to be situated in the vicinity of the vessels 11, 12, 13, and shown as an area of intersection 15 between the region of interest and the detection plane 14. The area of intersection 15 is shown cross-hatched in both images, FIG. 1a and FIG. 1b.

When the ultrasound apparatus is then moved, the region of interest, which is positionally defined relative to the ultrasound apparatus and in which the flow can be displayed clearly, also moves with it. A situation may arise such as is shown in FIG. 1b, in which the area of intersection 15 between the region of interest and the detection plane 14 no longer covers the entire region of the structures 11, 12, and 13, as is desired. In the present case, vessel 11 is outside the region of interest and vessel 12 is only partly within the region of interest, i.e., within the area of intersection 15. As a result, the flow of vessel 13 can be detected, but it is no longer possible to determine the flow of vessels 11 and 12.

In order to enable the flows to be detected again, the region of interest is manually changed or shifted in its position relative to the ultrasound apparatus. In doing so, the observation of the patient may be interrupted and the handling may be complicated. In attempts to avoid the need to manually change the ROI, the user may define a relatively large region of interest. With large regions, the structures to be displayed were still situated within these regions, even after a movement. This approach, however, has a disadvantage of slow image response time. When the regions of interest are larger, the frame rate of the ultrasound system may be significantly reduced, in particular with respect to the Doppler information, such to cause very slow image formation.

EP 1 041 395 B1 discloses a method for setting a region of interest in an image, wherein only the shape of the region of interest is changed when the depth or position is changed, to keep the size or number of scanning lines constant, and attempt to maintain the image response time.

U.S. Pat. No. 6,193,660 discloses determining the movement of the region of interest from a correlation between images obtained and shifting the region of interest accordingly, wherein the correlation is calculated on the basis of anatomical features or prominent image features (edges).

### SUMMARY OF THE INVENTION

A method in accordance with the present invention optimizes the display of an anatomical patient structure in a region of interest of a movable image detection apparatus. When the image detection apparatus is moved, the region of interest is shifted to detect particular properties of the patient structure to be observed. Shifting the region of interest may be minimized, whether performed manually or using image processing routines. Good image quality may be maintained as well as a fast frame rate, over the entire display time and the region of interest.

The method may include several steps, including one or more of the following:
a) Determining coordinates of a patient structure (which is contained in an image data set of a patient) to be displayed in a coordinate system that is spatially fixed or fixed relative to the patient.
b) Determining a region of interest (which includes the patient structure and in which a movable image detection apparatus can ascertain particular properties of the patient structure) in a coordinate system that is fixed relative to the movable image detection apparatus.
c) Tracking a change in the relative position between the movable image detection apparatus and the patient structure in the coordinate system that is spatially fixed or fixed relative to the patient using a medical tracking and/or navigation system.
d) Changing the position of the region of interest in the coordinate system of the image detection apparatus, such that the region includes the patient structure during and after the movement of the apparatus.
e) Displaying the patient structure using the image detection apparatus and an image output.

In other words, a method in accordance with the present invention may include navigation (i.e., determining and tracking the position of the image detection apparatus, to keep or place the region of interest at the correct point). Navigation and/or tracking systems are available in many treatment environments. Navigation reference devices are often provided to allow the navigation system and ultrasound apparatus to positionally integrate their images in an image-guided surgery procedure. Data from the ultrasound apparatus, when the ultrasound apparatus is tracked by the navigation system, can be correlated with previously produced image data sets (CT, MR, x-ray, etc.). For this correlation, the patient should be properly referenced and/or registered in the navigation environment.

In one embodiment, the region of interest is a volume of interest within a detection range of the image detection apparatus, and the region of interest is assigned a defined position within a patient coordinate system that is spatially fixed or fixed relative to the patient. The region of interest of the image detection apparatus can be defined manually at the beginning of the procedure or during display. In preoperative planning, a user may fix a starting point for the region of interest, using a user interface or other hardware or software.

When the image detection apparatus is moved while an image is being displayed, the detection apparatus' parameters can guide, shift, or adjust the region of interest within in the coordinate system of the image detection apparatus, in accordance with the movement.

The region of interest of the image detection apparatus may be defined using a user interface or other software, at the beginning of a procedure or while an image is being displayed. Software in accordance with the invention, may automatically define the region of interest, in a section that includes the patient structure and is to be displayed.

The section to be displayed does not have to be a stationary section in a patient structure. The section can be shifted while the image is being displayed (in particular along the patient structure), wherein the region of interest of the image detection apparatus can be guided, shifted, or adjusted in accordance with the movement of the section to be displayed.

In another embodiment, the size and/or shape of the region of interest may be changed, and may be adjusted to the patient structure.

In another embodiment, an ultrasound image detection apparatus may be used as the image detection apparatus. A Doppler ultrasound apparatus may be selected for detecting flow properties (e.g., flow velocities) in patient vessels (e.g., blood vessels). With such equipment, it is possible to determine an angular position of the vessel relative to an image detection plane of the ultrasound image detection apparatus from a sectional geometry of a sectional image of the vessel. Additionally, it is possible to correct the ascertained data concerning the flow properties (flow velocity) in accordance with the angular position.

The image detection apparatus used in performing the method herein is not limited to an ultrasound apparatus. The image detection apparatus can be any image detection apparatus in which a “region of interest” can be defined. Examples of appropriately equipped image detection apparatus include: computer tomographs, nuclear spin tomographs, x-ray image detection apparatus, and the like.

### BRIEF DESCRIPTION OF THE DRAWINGS

The forgoing and other features of the invention are hereinafter discussed with reference to the figures.

FIGS. 1a and 1b (as described above) illustrate a shifting of the region of interest, as performed in methods in accordance with the prior art.

FIG. 2 illustrates using an exemplary ultrasound device in connection with an exemplary medical navigation system to perform so-called “navigated ultrasound integration.”

FIG. 3 depicts a region of interest of an ultrasound probe in a coordinate system that is spatially fixed or fixed relative to the patient.

FIGS. 4a and 4b illustrate guiding and/or shifting the region of interest in accordance with the movement of the image detection apparatus.

FIG. 5 schematically shows an exemplary data processing device, or computer, in accordance with the present invention

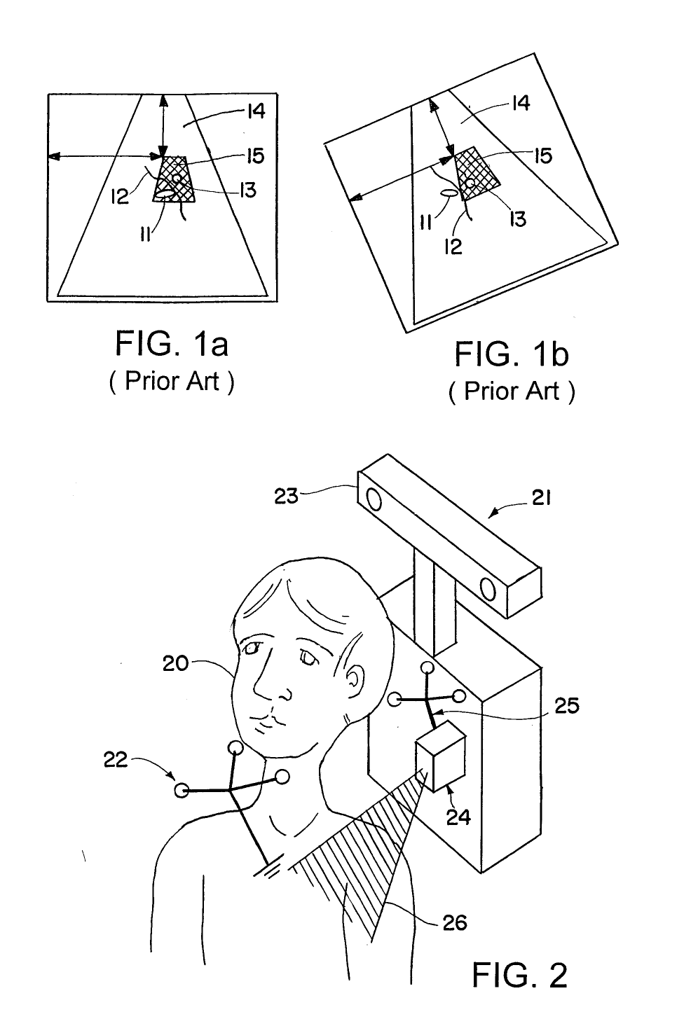

### DETAILED DESCRIPTION

Image-guided surgery or treatment often relies upon a navigation system to track the patient and various medical instruments to provide useful information to the physician. In an example embodiment shown in FIG. 2, an ultrasound device is used in connection with the navigation system to perform so-called “navigated ultrasound integration.” In such a procedure, a patient 20 is “registered” so that a navigation system 21 “knows” the patient's position and, when the patient 20 has a reference array 22 attached, the navigation system 21 can track the patient's movement using a sensor array 23. In the present example, an ultrasound device 24 is equipped with a reference array 25 so that the navigation system 21 can detect the ultrasound device's position and can track its movement. Like the patient 20 the ultrasound device 24 may be registered or “calibrated” such that the navigation system 21 knows the position of the ultrasound device's image detection plane 26 relative to the reference array 25. Therefore, each time the ultrasound device 24 records an image, the navigation system 21 “knows” the position of the image with respect to one or more defined coordinate systems.

The position information associated with the ultrasound image may be used to define a region of interest that may be “fixed” in a coordinate system of the patient 20. The patient coordinate system may be fixed to the patient 20 and moves when the patient moves. In this example, the ultrasound device 24 is an ultrasound probe calibrated such that, in terms of spatial relation, ultrasound image coordinates are assigned and known in the patient or “global” coordinate system.

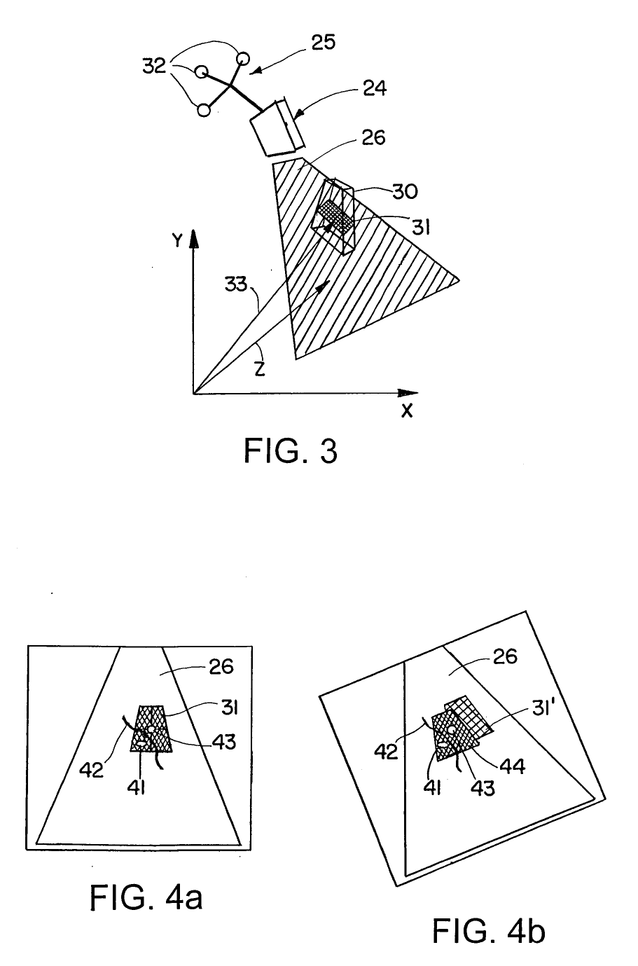

The region of interest can be a three-dimensional, box-like region 30 shown in FIG. 3. FIG. 3 also shows an ultrasound probe 24 and its respective image detection plane 26. The region of interest 30 can be a region that its position is initially fixed in relation to the ultrasound probe 24, and in the case of Doppler ultrasound, it is a region where specific flow properties, such as flow velocities, can be reproduced in color. An area of intersection 31 between the region of interest 30 and the detection plane 26 of the ultrasound probe 24 is cross-hatched in FIG. 3.

As noted above, the ultrasound probe 24 may be used in connection with a navigation system, and can be tracked in one or more assigned or defined coordinate systems. In the example shown in FIG. 3, the ultrasound probe 24 is equipped with a reference array 25, and the probe's position can be tracked by a navigation system (not shown). In this example, the reference array 25 may tracked via three reflective markers 32. The probe's position may be determined in a patient coordinate system x, y, z, which is spacially fixed to the patient.

A position of the center point of the patient region of interest 30 is defined by a vector 33 in the patient coordinate system x, y, z. The region of interest 30 can be initially defined to be positionally fixed relative to the ultrasound probe 24. Therefore, the region of interest 30 is moved in the patient coordinate system x, y, z (which is spatially fixed or fixed relative to the patient) when the ultrasound probe 24 moves. The region of interest 30, however, remains at the same point in a coordinate system u, v, w (not shown) fixed relative to the ultrasound probe 24.

The three-dimensional region of interest 30 (volume of interest) has a defined position in the patient coordinate system x, y, z which is spatially fixed or fixed relative to the patient. The center of mass of the region of interest 30 can be placed within the region of the anatomical patient structure to be displayed (for example, on a part of a vessel) and can remain on this position.

Turning now to FIGS. 4a and 4b, the above situation is illustrated. FIG. 4a shows the initial state in which the area of intersection 31 between the image detection plane 26 and the region of interest 30 lies over the blood vessels 41, 42, and 43. Upon moving the probe, the area of intersection 31 is shifted away from the vessels 41, 42, and 43, as shown by the more largely cross-hatched region 31′ in FIG. 4b. The movement of the probe 24 can be tracked and quantified by the navigation system using the reference array 25, and the region of interest can be correspondingly shifted such that its area of intersection 44 with the detection plane 26 again lies within the region of the vessels 41, 42, 43. The settings for the region of interest in the ultrasound hardware/software thus track the movement and shift the region of interest back onto the patient structures to be displayed (the blood vessels 41, 42, 43). Optionally, the center of the region of interest 30 can be automatically set along a patient structure (for example, a segmented blood vessel) or can be automatically set on the basis of any other information from previously acquired data (CT, MRI, etc.). The automatically set center of the region of interest 30 may always be kept at the current image position of the selected patient structure. The center of the region of interest 30 also can be marked (as a landmark point), either manually before, or during the examination, or automatically detected using a segmented patient structure that lies within the ultrasound detection plane 26.

The method in accordance with the invention can use information from the navigation system to calculate the coordinates and/or new coordinates of a selected region of interest in a coordinate system that is spatially fixed or fixed relative to the patient. These coordinates can be calculated using the calibration information of the reference array equipped ultrasound probe.

The coordinates of the region of interest are “fixed” with respect to the orientation of the patient and define the region of interest. This region or volume may be a box-like or otherwise configured three-dimensional shape, and a center of mass of the three-dimensional shape is fixed at its calculated position in a patient coordinate system. When the navigated probe is moved in the patient coordinate system or patient space, the current settings for the region of interest of the probe are changed, using the information concerning the area of intersection between the image detection plane and the “fixed” volume of interest. Therefore, during ultrasound imaging an initially selected blood vessel may remain in the region of interest, as long as it is visible somewhere in the ultrasound image.

The shape of the region of interest and/or volume of interest can be adjusted for a number of applications. It could have a greater depth, to be able to better follow an anatomical structure to be displayed. Alternatively, the region of interest can be defined by following a particular anatomical structure (for example, a blood vessel). In this example, the region of interest of the ultrasound image may be selected based on the point of intersection between the vessel structure and the image detection plane, wherein a region around the point is selected in which the vessel is visible in the image. The point of intersection between the vessel and the image detection plane can be defined by a segmented object from vessel recognition in the previously acquired image data set, or by any other object from the pre-operative treatment planning.

In the case of Doppler imaging using an ultrasound device, the method can assist the user, particularly if the angle of the moving fluid with respect to the sound beam has to be taken into account by the user, to correctly detect the flow velocity. If a predefined vessel object is used to set the region of interest, use of the method can define the angle using the sectional geometry of the vessel in the image detection plane, and can store this information in the ultrasound device's memory. In this manner, the user can determine a correct indication of the velocity, without an additional intervention.

Overall, the method in accordance with the invention allows the user to concentrate on the examination while he or she freely moves the image detection apparatus (probe). No longer does the user have to spatially restrict the movements of the image detection apparatus to ensure the correct position of the region of interest (for example, a flow window). Moreover, the user does not have to adjust the region of interest every time he or she changes the position or angle of the image detection apparatus. The method in accordance with the invention also allows the user to set a relatively small region of interest, which enables a high frame rate and better and faster ultrasound image detection.

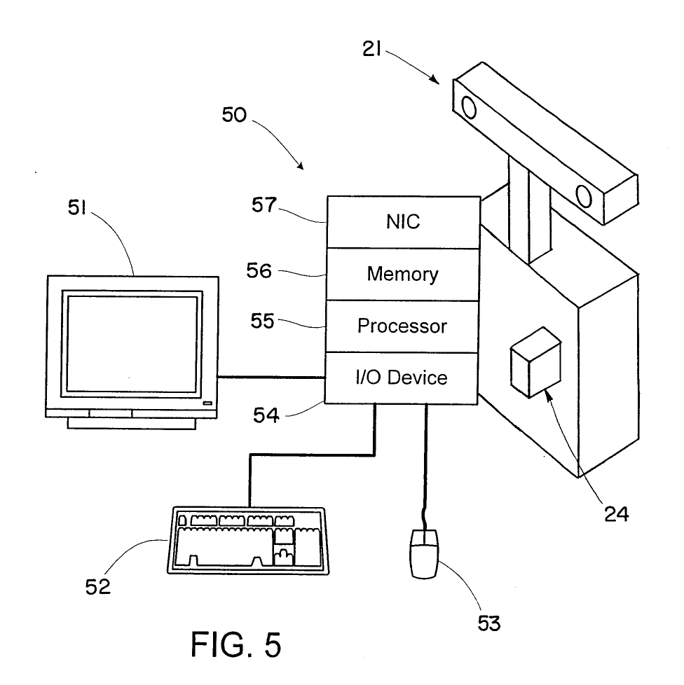

Moving now to FIG. 5 there is shown a block diagram of an exemplary data processing device or computer 50 that may be used to implement one or more of the methods described herein. The computer 50 may be a standalone computer, or it may be part of a medical navigation system, for example. The computer 50 may include a display 51 for viewing system information, and a keyboard 52 and pointing device 53 for data entry, screen navigation, etc. A computer mouse or other device that points to or otherwise identifies a location, action, etc., e.g., by a point and click method or some other method, are examples of a pointing device 53. Alternatively, a touch screen (not shown) may be used in place of the keyboard 52 and pointing device 53. The display 51, keyboard 52 and mouse 53 communicate with a processor via an input/output device 54, such as a video card and/or serial port (e.g., a USB port or the like).

A processor 55, such as an AMD Athlon 64® processor or an Intel Pentium IV® processor, combined with a memory 56 execute programs to perform various functions, such as data entry, numerical calculations, screen display, system setup, etc. The memory 56 may comprise several devices, including volatile and non-volatile memory components. Accordingly, the memory 56 may include, for example, random access memory (RAM), read-only memory (ROM), hard disks, floppy disks, optical disks (e.g., CDs and DVDs), tapes, flash devices and/or other memory components, plus associated drives, players and/or readers for the memory devices. The processor 55 and the memory 56 are coupled using a local interface (not shown). The local interface may be, for example, a data bus with accompanying control bus, a network, or other subsystem.

The memory may form part of a storage medium for storing information, such as application data, screen information, programs, etc., part of which may be in the form of a database. The storage medium may be a hard drive, for example, or any other storage means that can retain data, including other magnetic and/or optical storage devices. A network interface card (NIC) 57 allows the computer 50 to communicate with other devices.

A person having ordinary skill in the art of computer programming and applications of programming for computer systems would be able in view of the description provided herein to program a computer system 50 to operate and to carry out the functions described herein. Accordingly, details as to the specific programming code have been omitted for the sake of brevity. Also, while software in the memory 56 or in some other memory of the computer and/or server may be used to allow the system to carry out the functions and features described herein in accordance with the preferred embodiment of the invention, such functions and features also could be carried out via dedicated hardware, firmware, software, or combinations thereof, without departing from the scope of the invention.

Computer program elements of the invention may be embodied in hardware and/or in software (including firmware, resident software, micro-code, etc.). The invention may take the form of a computer program product, which can be embodied by a computer-usable or computer-readable storage medium having computer-usable or computer-readable program instructions, “code” or a “computer program” embodied in the medium for use by or in connection with the instruction execution system. In the context of this document, a computer-usable or computer-readable medium may be any medium that can contain, store, communicate, propagate, or transport the program for use by or in connection with the instruction execution system, apparatus, or device. The computer-usable or computer-readable medium may be, for example but not limited to, an electronic, magnetic, optical, electromagnetic, infrared, or semiconductor system, apparatus, device, or propagation medium such as the Internet. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner. The computer program product and any software and hardware described herein form the various means for carrying out the functions of the invention in the example embodiments.

Although the invention has been shown and described with respect to a certain preferred embodiment or embodiments, it is obvious that equivalent alterations and modifications will occur to others skilled in the art upon the reading and understanding of this specification and the annexed Figures. In particular regard to the various functions performed by the above described elements (components, assemblies, devices, software, computer programs, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for displaying an anatomical patient structure in a region of interest of a movable image detection apparatus, comprising:
a) determining a position of the patient structure in a patient coordinate system;
b) defining a position of a region of interest in an image detection apparatus coordinate system wherein the region of interest includes the patient structure;
c) using a medical tracking system to track position changes between the image detection apparatus relative to the patient structure in the patient coordinate system;
d) changing the defined position of the region of interest in the image detection apparatus coordinate system in accordance with the position changes between the image detection apparatus relative to the patient structure, such that the defined region of interest includes the patient structure; and
e) displaying the patient structure and/or properties of the patient structure.

2. The method according to claim 1, wherein the region of interest includes the patient structure during and after the position changes between the image detection apparatus relative to the patient structure.

3. The method according to claim 1, wherein the region of interest is a volume of interest.

4. The method according to claim 1, further comprising manually defining a size and/or position of the region of interest at the beginning of a procedure.

5. The method according to claim 1, wherein when the image detection apparatus is moved while the image is being displayed, the defined position of the region of interest is changed in accordance with the tracked movement of the image detection apparatus.

6. The method according to claim 1, further comprising automatically defining the region of interest of the image detection apparatus via image processing.

7. The method according to claim 1, further comprising:
shifting a section that is to be displayed along the patient structure within the patient coordinate system, and
changing the region of interest of the image detection apparatus in accordance with the shifting of the section to be displayed.

8. The method according to one claim 1, further comprising adjusting a size and/or shape of the region of interest in accordance with a size and/or shape of the patient structure.

9. The method according to claim 1, wherein the image detection apparatus is a Doppler ultrasound apparatus for detecting flow properties.

10. The method according to claim 9, further comprising:
determining an angular position of a through-flow vessel relative to an image detection plane of the ultrasound apparatus; and
using the determined angular position to calculate and/or correct flow properties of the through-vessel.

11. A computer program embodied on a computer readable medium for displaying an anatomical patient structure in a region of interest of a movable image detection apparatus, comprising:
a) code that determines a position of the patient structure in a patient coordinate system;
b) code that defines a position of a region of interest in an image detection apparatus coordinate system wherein the region of interest includes the patient structure;
c) code that use a medical tracking system to track position changes between the image detection apparatus relative to the patient structure in the patient coordinate system;
d) code that changes the defined position of the region of interest in the image detection apparatus coordinate system in accordance with the position changes between the image detection apparatus relative to the patient structure, such that the defined region of interest includes the patient structure; and
e) code that displays the patient structure and/or properties of the patient structure.

12. A system for detecting an anatomical patient structure in a region of interest of a movable image detection apparatus, comprising:
an image detection apparatus equipped with a reference array;
a reference array configured for attachment to a patient;
a navigation system configured to spatially track the reference arrays;
a display device; and
a computer operatively coupled to said navigation system, said image detection device, and said display device, said computer comprising
a processor and memory, and
logic stored in the memory and executable by the processor, said logic including
i) logic that determines a position of the patient structure in a patient coordinate system;
ii) logic that defines a position of a region of interest in an image detection apparatus coordinate system wherein the region of interest includes the patient structure;
iii) logic that uses a medical tracking system to track position changes between the image detection apparatus relative to the patient structure in the patient coordinate system;
iv) logic that changes the defined position of the region of interest in the image detection apparatus coordinate system in accordance with the position changes between the image detection apparatus relative to the patient structure, such that the defined region of interest includes the patient structure; and
v) logic that displays the patient structure and/or properties of the patient structure.
